(19) United States
(12) Patent Application Publication
(10) Pub. No.: US 2010/0070031 A1
(43) Pub. Date: Mar. 18, 2010

(54) Artificial joint

(76) Inventor: Alfred Buck, Bondorf (DE)

Correspondence Address: DeMont & Breyer, LLC, 100 Commons Way, Ste. 250, Holmdel, NJ, 07733, US

(21) Appl. No.: 12/536,847

(22) Filed: Aug. 6, 2009

(30) Foreign Application Priority Data
Aug. 11, 2008 (DE) 10 2008 037 201.3

(51) Int. Cl. A61F 2/02

(52) U.S. Cl. 623/11.11

(57) ABSTRACT

The present invention relates to an implant for surgical use in humans or vertebrate animals in the replacement, partial replacement or reinforcement of a joint or an intervertebral disk and to a method for the production of an implant. In a first embodiment, the implant comprises a composite structure of a first section, which is formed at least by one compression-molded component made of wire, and a second section, with the first section being at least in parts embedded in the second section. In a second embodiment, the implant also comprises a compression-molded component. This component is formed by a wire made of a first material, with parts of said wire of the molded component having a coating made of a second material.

### FIELD OF INVENTION

The present invention relates to an implant for surgical use in humans or vertebrate animals in the replacement, partial replacement or reinforcement of a joint or an intervertebral disk and to a method for the production of an implant.

### BACKGROUND OF THE INVENTION

Joints are connecting areas between bones and serve to enable the body to move. In addition to ensuring mobility, the joints at the same time also play a role in the absorption and compensation of the pressure exerted on the skeletal system, which pressure builds up while standing or jumping.

Because of diseases and/or wear and tear, it may be necessary to replace the natural joints with artificial joints which are implanted as implants into the bones. Such a joint replacement may, for example, involve a shoulder, hip or knee joint, the articular head and the joint socket of which are replaced with implants made of a plastic material which must be solidly anchored in the bone. In this context, it should be noted that a distinction is made between a total endoprosthesis where both the articular head and the joint socket are replaced and the partial endoprosthesis in which only the articular head is replaced.

Generally, the implants are, for example, anchored in a bone either by allowing them to grow into the bone or tissue and/or by cementing them in. In some cases, the implants can also be anchored in the bone by means of screws. As a rule, acetabula, for example, are screwed into the bone.

To enable a movement that is unrestricted within the confines of the natural movement, the implant is generally adapted to conform to the properties of the joint that is to be replaced, such as to the dimensions, shape and the static and/or dynamic properties. In this context, dynamic properties refer especially to the rigidity, elasticity, springiness and/or damping of the implant.

DE 10 2004 041 354 A1 discloses an implant in which a mesh fabric is used to replace the springy or elastic part of a joint or intervertebral disk. The mesh fabric itself is made of a wire mesh. The disclosure of the application mentioned, in particular the implant described and the method of producing the implant, is by reference fully incorporated into the present invention. The porosity of the compression-molded mesh fabric makes it possible for the implant to unite with the bone and/or the tissue by allowing the bone and/or the tissue to grow into the implant.

If, however, the wire described in the patent mentioned is a so-called bioinert material, for example, titanium, it is possible for the bone and/or the tissue to grow far enough or even completely into the implant so that the implant is no longer able to meet the requirements, in particular with respect to its dynamic properties. In such a case, this may even lead to an undesirable rigidity of the implant.

In addition, the surfaces of the implant that are moved relative to each other must also meet stringent requirements with respect to a good sliding property while ensuring minimum friction. Furthermore, the formation of abraded material should be avoided as much as possible.

### SUMMARY OF THE INVENTION

The problem to be solved by the present invention is to make available an implant and a method of producing an implant which at least reduce the prior-art disadvantages described above.

Specifically, it should be possible to improve already existing implants in such a manner that both an improved anchorage of the implant in the tissue and/or the bone and an improved functionality of the joint are made available.

In a first embodiment, the present invention claims an implant for surgical use in humans or vertebrate animals in the replacement, partial replacement or reinforcement of a joint or an intervertebral disk, comprising a composite structure of
a first section which is preferably formed by at least one compression-molded component made of wire and
a second section, with the first section, preferably the molded component, being at least in parts embedded in the second section.

The scope of the present invention also covers a method for the production of an implant for use in the replacement, partial replacement or reinforcement of a joint or an intervertebral disk, in particular an implant, which method comprises the following steps:
making available a mesh fabric made of wire,
folding and/or rolling up the mesh fabric,
compressing the mesh fabric into a compression-molded component for forming a first section,
making available a second section and
connecting the first section with the second section, with the first section, preferably the molded component, being at least partially or partially embedded in the second section.

The method is especially well suited to the production of an implant as disclosed by the present invention. The first section and second section can also be referred to as the first and second structure.

A compression-molded component is a component which is given its desired shape by means of compression molding. The shape of the molded component is substantially created by means of the compression molding method. The molded component is primarily responsible for the dynamic properties. For details of the production of the compression-molded component, reference is hereby made to the description below.

Since the compression-molded component is made of wire or consists of wire, it is a porous structure. This porous structure makes it possible for the molded component or the wire of the molded component to be at least partially or partially embedded in the second section. In this context, embedding is defined to mean that, on the one hand, the wires of the molded component have penetrated into the volume or at least into the surface of the second section wherein they are anchored. Embedding causes part of the hollow spaces of the porous molded component to be filled or partially filled with the material of the second section. To obtain a stable anchorage and to maintain the dynamic properties intact, the lateral surface of the molded component is embedded up to one half, preferably up to one third, especially up to one fifth of its thickness in the second section. In another embodiment of the present invention, the first structure, preferably the molded component, and the second structure are fusion-bonded to each other.

The second section as such is preferably not a compression-molded component made of wire. It has properties different from those of the first section or compression-molded component. The second section has a lower porosity than the molded component. The second section is preferably solid, i.e., not porous. In one embodiment, the second section is made of a nonmetal material.

The second section is or contains at least one material from the group comprising ceramics and plastics. At least certain parts of the second section have a low viscosity to such an extent that the first section, preferably the molded component, can be embedded or received [in the second section]. This can generally be implemented by heating the material of the second section so that the material becomes moldable or, for example, even “liquid.” The stable anchorage is achieved by solidifying the second section.

In a first embodiment, the second section can be made directly available as a structure that is subsequently connected with the first section. In a second embodiment, the second section has been formed or is being formed on top of the first section, preferably the molded component. For example, if the implant is made of a plastic material, to form the second section, the material is sprayed onto the first section, preferably the molded component. For example, if the implant is a ceramic material, to form the second section, the material is sintered onto the first section, preferably the molded component.

Especially when a joint socket or an articular head is to be created, the first section, preferably the molded component, is configured in the form of a first shell with an inside surface and an outside surface. Depending on the type of implant that is to be created, the second section is disposed on the inside surface of the first section or on the outside surface of the first section. The second section is preferably also configured in the form of second shell with an inside surface and an outside surface. In one embodiment, the first shell and the second shell are nested one in the other or adjoin each other. The inside space of the second shell preferably forms the receiving space for the articular head. Thus, the articular head slides along the inside surface of the second shell.

The second section is primarily responsible for the sliding properties of the implant. However, it can also contribute to the dynamic properties of the implant. In a preferred embodiment, the second section is disposed on a lateral surface of the implant, which lateral surface forms a sliding plane for a movement in a joint. Thus, the second section forms the sliding plane or a sliding plane of the implant. In the case of an articular head, this plane is the upper surface or the outside surface of the articular head. In the case of a joint socket, this plane is the lower surface or the outside surface of the joint socket.

The first section, preferably the molded component, on the other hand, is disposed on a lateral surface of the implant, which lateral surface adjoins the surrounding tissue or the adjoining bone to which the implant is to be connected or anchored. The first section, preferably the molded component, forms the connecting element. In the case of an articular head, this is the lower surface or the inside surface of the articular head. In the case of a joint socket, this is the upper surface or the inside surface of the joint socket.

In one embodiment of the invention the compression-molded component, preferably the wire of the compression-molded component, has at least in parts a coating. In detail, the wire of the molded component is made of a first material, with the wire of the molded component at least in parts or in parts having a coating made of a second material.

Preferably this contains the step of applying a coating, in particular a partial coating, to the wire with a second material, with the first material or the second material being a material which, after implantation of the implant, makes it possible, in a boundary region relative to a surrounding tissue or bone, for the tissue to grow at least in parts into or to develop at least in a surface region of the compression-molded component.

The scope of the present invention also covers an implant for surgical use in humans or vertebrate animals in the replacement, partial replacement or reinforcement of an intervertebral disk or a joint, comprising a compression-molded component which is formed by wire made of a first material, with the wire of the molded component at least in parts or in parts having a coating made of a second material.

In addition, the present invention also claims a method for the production of an implant for use in the replacement, partial replacement or reinforcement of a joint or an intervertebral disk, which method comprises the following steps:

making available a mesh fabric that is made of wire of a first material,

folding and/or rolling up the mesh fabric,

compression molding the mesh fabric into a molded component, and

applying a coating, in particular a partial coating, to the wire with a second material, with the first material or the second material being a material which, after implantation of the implant, makes it possible, in a boundary region relative to a surrounding tissue or bone, for the tissue to grow at least in parts into or to develop at least in a surface region of the compression-molded component. The method is especially useful to produce an implant according to the present invention.

As already mentioned above, certain materials, for example, titanium, tend to grow into the tissue, such as a bone and/or cartilage, or make if possible for the tissue to grow into an implant or a porous structure. In some cases, this can cause the implant to lose its dynamic properties and thus its function. This potential disadvantage, however, can be turned into an advantage since an extremely strong connection forms between the implant and the tissue of the structure.

By specifically identifying those areas of the implant or molded component on which ingrowth or anchorage is desirable, it is possible to create a strong bond with the neighboring or adjoining tissue, on the one hand, and at the same time, maintain the dynamic properties of the implant intact, on the other hand.

The decision of whether to use the first material or the second material with the “connecting” material depends on which material forms the area adjoining the surrounding tissue or bone so that a stable anchorage to the tissue or bone can be formed.

Ingrowth or anchorage should preferably be made possible in the boundary region between the tissue and the implant. On an implant in the form of an intervertebral disk, this is the upper surface and/or the lower surface of the intervertebral disk. In this case, the compression-molded component has the shape of a kidney with a curved convex surface and an oppositely lying concave surface. On an implant in the form of a joint socket or an articular head, this is the posterior surface of the joint socket or articular head. In this case, the molded component can have the shape of a shell.

In one embodiment of the present invention, prior to shaping the compression-molded component, the wire, in accordance with its function, is treated in those areas which, after the production of the molded component, form the desired segment of the implant.

Thus, for example, if the material used for the wire, such as titanium and/or its alloys, promotes ingrowth of the tissue, the wire is coated in parts or throughout with a coating that largely reduces or suppresses ingrowth. An example of such a material is a plastic, for example, silicone and/or Teflon, and/or a precious metal, for example, gold.

If, instead, the material used for the wire suppresses ingrowth of the tissue, the wire is coated in parts or throughout with a coating that largely promotes or enables ingrowth. As already mentioned above, an example of such a material is titanium and/or its alloys.

In a preferred embodiment of the present invention, the wire, in particular the wire itself or the coated wire, in accordance with its function, is treated, only after the molded component has been produced, in those areas which, after the production of the molded component, form the desired segment of the implant. Preferably ingrowth should be enabled at least in parts in a boundary region between the tissue and the implant.

If the wire is coated with a coating that does not enable ingrowth, the boundary region of the implant can be treated in such a manner that ingrowth is enabled at least in a surface region. This can be implemented by removing the coating. In one embodiment, the coating is removed by etching. To this end, the implant can be simply immersed [into the etching medium]. The properties in the boundary region are determined by the depth and/or the length of immersion. Thus, in one embodiment, at least one of the surfaces of the molded component is not coated with a coating, which surface, after implantation of the implant, borders on surrounding tissue so that the tissue is able to grow especially into a surface region of the molded component. To this end, after compression molding the molded component, the coating is removed at least on one surface of the molded component so that the surface of the wire is exposed.

If the wire is coated with a material that does not allow ingrowth, the boundary region of the implant can be treated to ensure that ingrowth is enabled at least in a surface region. This treatment can be done by applying a coating to the wire. In one embodiment, the wire is electroplated. To this end, the implant can simply be immersed into the bath. The properties in the boundary region are determined by the depth and/or the length of immersion. Thus, in another embodiment, at least one of the surfaces of the molded component is not coated, which surface, after implantation of the implant, borders on surrounding tissue, thus enabling ingrowth of the tissue, in particular into a surface region, of the molded component. To this end, after compression-molding the molded component, the coating is removed at least on one side of the molded component so that the surface of the wire is exposed.

If the wire is made of a material which does not permit ingrowth, the boundary region of the implant can be treated in such a manner that ingrowth is possible at least in one surface region. This treatment can be done by applying a coating to the wire. In one embodiment, the wire is electroplated. To this end, the implant can simply be immersed into the bath. The properties in the boundary region are determined by the depth and/or the length of immersion. Thus, in another embodiment, the implant, after having been implanted, is coated in a boundary region relative to surrounding tissue so that ingrowth of the tissue, preferably at least in one surface region, is possible. This coating on at least this one surface of the molded component is applied after the component has been compression-molded.

To prevent excessively deep ingrowth of the tissue, the wires of the molded component which form the core of the molded component are left untreated or are coated with a coating which prevents the tissue from growing into the core of the molded component.

The first material or the second material is or comprises a largely bioinert and/or bioactive material. The properties of the first material differ from those of the second material.

Bioinert materials include, for example, titanium, aluminum and/or zirconium. It is assumed, without however being restricted to this theory, that contact osteogenesis occurs. When this happens, a bioinert material is surrounded by the tissue or a fibrous connective tissue. It appears to be some type of a morphological fixation in which nonporous inert materials unite with the surrounding tissue in that the bone grows into the uneven parts of the surface. The tissue friendliness of titanium or its alloys is probably due to an existing oxide layer. This oxide layer separates the implant from the surrounding tissue or connective tissue. Thus, a direct anchorage to the bone is made possible. This also seems to apply to aluminum and aluminum oxide and/or zirconium and zirconium oxide.

Bioactive materials include, for example, glass ceramics and/or hydroxyl apatite. It is assumed, without however being bound by this theory, that bonding osteogenesis occurs. Bioactive material bonds directly with the surrounding tissue. The stable connection is probably due to physical and chemical bonding with the bone.

The following discussion relates primarily to the compression-molded component as such. This component is made of wire. Depending on the requirements that must be met, the wire is generally made of or generally comprises titanium, a titanium alloy, stainless steel or a stainless steel alloy. The wire has a diameter of approximately 0.01 mm to 5 mm, preferably approximately 0.05 mm to approximately 1 mm, especially preferred is a diameter of approximately 0.2 to approximately 0.3 mm.

In one embodiment of the present invention the compression-molded component is made of a mesh fabric. To obtain a regular structure, the mesh fabric is preferably a knit fabric. In a preferred embodiment, the knit fabric is a circular-knit fabric. This makes it possible to effectively avoid edges and the formation of potential inhomogeneities in the molded component. In addition, the mesh fabric can also be embossed.

The compression-molded component is made in particular from a folded and/or rolled-up mesh fabric. By folding and/or rolling, it is possible to adjust the dynamic properties, in particular by way of the density of the mesh fabric. The mesh fabric preferably is first folded and then rolled up.

The compression molding procedure or the shape-imparting step to create the molded component is carried out by means of a hydraulic or pneumatic compression mold in a negative mold which conforms to the shape of the molded component.

The molded component obtained has a degree of porosity between 20% and 80%, preferably between 30% and 70%, especially preferred is a porosity between 20% and 60%. The molded component has a mesh width of 0.01 mm to 50 mm, preferably 0.5 mm to 20 mm, especially preferred is a mesh width of 3 mm to 8 mm. The implant or the molded component is at least in parts elastic. It has an elasticity constant which, in particular initially, is in a range from 50 to 3000 N/m, preferably from 100 to 1000 N/mm, and especially from 150 to 800 N/mm.

### BRIEF DESCRIPTION OF THE DRAWINGS

The present invention will be explained in greater detail based on the following practical examples. To this end, reference is made to the appended drawings. Identical reference numerals in the various drawings refer to the same elements.

FIG. 1 shows a schematic view of the configuration of an endoprosthesis for a hip joint.

FIGS. 2.a and 2.b show a schematic perspective view of a cross section through the acetabulum seen in FIG. 1.

FIGS. 3.a and 3.b show a schematic view of a cross section through an acetabular implant according to the present invention.

FIGS. 4.a to 4.c show, respectively, a lateral view, a bottom view and a perspective view of a detailed illustration of a molded component for an acetabulum (without the second section).

FIGS. 5.a and 5.b illustrate a section of a vertebral column and an intervertebral disk implant.

FIGS. 6.a and 6.b show a schematic view each of a cross section through an intervertebral disk implant according to the present invention.

FIG. 7 shows a detailed view of an intervertebral disk implant according to the present invention.

### DETAILED DESCRIPTION

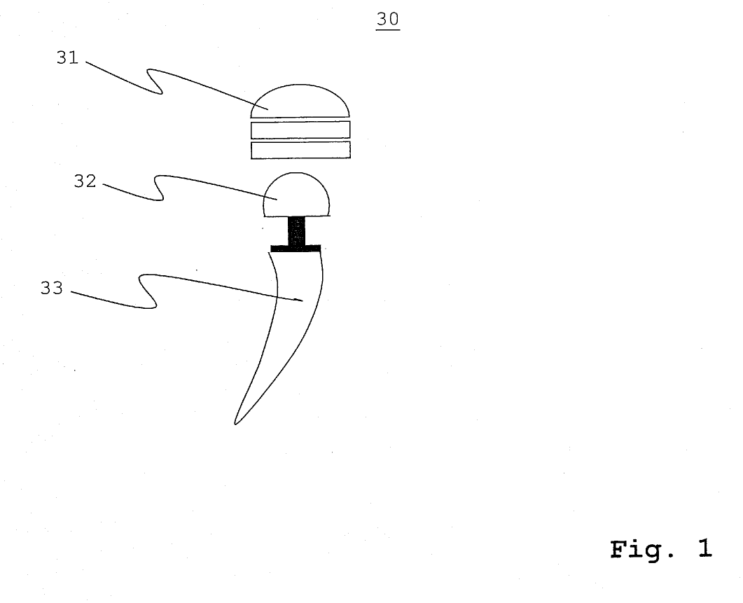

FIG. 1 shows a schematic view of the configuration of an endoprosthesis for a hip joint 30. It is a total endoprosthesis with which both the acetabulum 31 and the articular head 32 are replaced. The articular head 32 can be stably anchored in the bone, for example, by means of a stem 33. The hip joint 30 is a type of ball-and-socket joint. The ball end of one section 32 meshes with the hollow spherical cavity of the other section 31, which preferably makes a swiveling movement in all directions possible. The invention will be explained using a hip joint 30 as an example. It can, however, also be used in any other joint, for example, the shoulder, hip and/or knee joint.

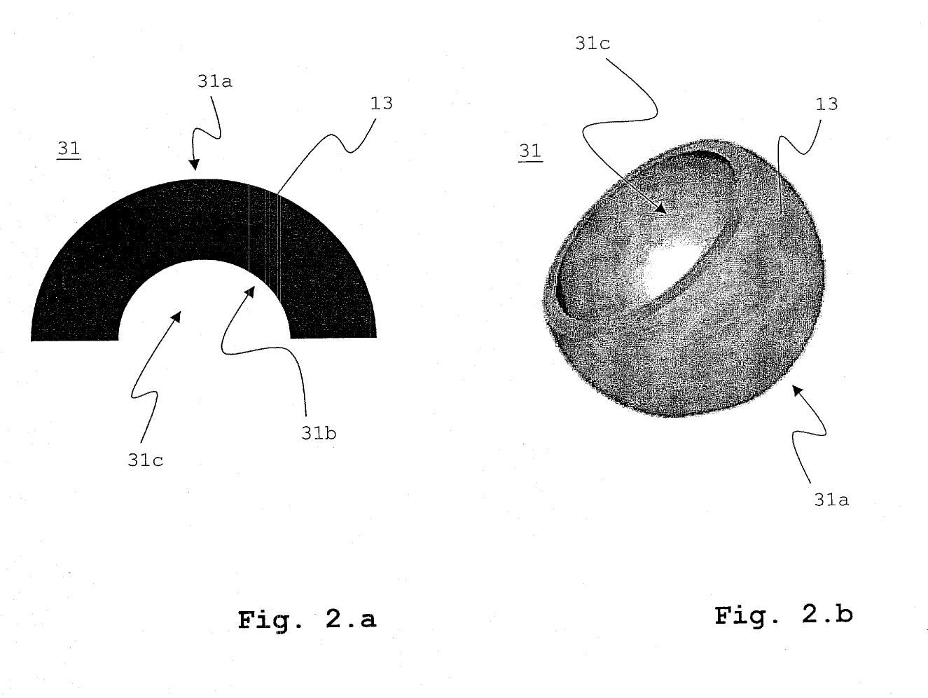

FIG. 2.a shows a schematic view of a cross section through the acetabulum 31 seen in FIG. 1. Known acetabula 31 frequently are made of specialty plastics or ceramic materials. The acetabulum 31 is designed as a type of shell 13 or hemispherical shell 13. A hemispherical shell is the set difference between substantially concentric hemispheres with different radii. This is once more illustrated in a perspective view in FIG. 2.b. The acetabulum 31 is connected to the bone 50 or to the tissue 50 by way of its outside surface 31a. At least parts of the inside space 31c of the acetabulum 31 receive the articular head 32. The movement of the articular head 32 is, so to speak, guided by the acetabulum 31. The inside surface 31a of the acetabulum 31 and the outside surface of the articular head 32 (not shown in this figure) are adjoining each other. These surfaces are generally smooth to the point that a sliding movement with minimum friction, preferably without the generation of abrasive material, can be ensured.

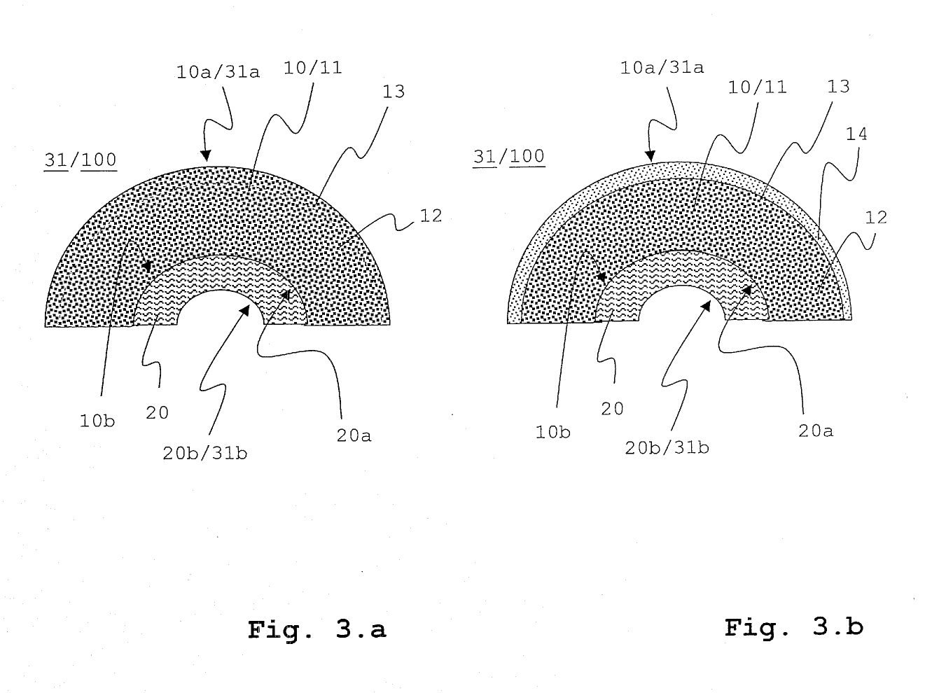

FIG. 3a shows a schematic view of a cross section through an implant 100 for an acetabulum 31 according to the present invention. The implant 100 comprises a plurality of sections or a plurality of structures. As shown, the implant 100 or the acetabulum comprises or consists of a compression-molded component 11 made of wire 12, in particular of a molded component 11 made from a compression-molded mesh fabric made of wire 11, as the first section 10 and of a plastic component as the second section 20.

The second section 20 is disposed in the inside space of the first section 10 or the molded component 11. The molded component, so to speak, embraces the second section 20. The outer surface 20a of the second section 20 preferably rests against the inside surface 10b of the molded component 11. To ensure a stable and permanent anchorage, the first section 10 and the second section 20 are fusion-bonded to each other.

In the embodiment mentioned in which the second section 20 is made of a plastic material, this section is preferably applied or sprayed onto the first section 10 or molded component 11. In this case, the molded component 11, or more specifically the inside surface 10b of the molded component 11, constitutes a type of negative mold. The second section 20 should essentially make it possible for an articular head 32 to perform a sliding movement in the acetabulum 31.

In contrast, the molded component 11 is primarily responsible for the dynamic properties of implant 100 and to make possible a stable anchorage in the surrounding tissue 50 or bone 50. The outside surfaces 10a of implants 100 that are facing the bone 50 or tissue 50 are porous since the molded component 11 is made from a compression-molded mesh fabric. The porosity of the molded component 11 makes it possible for the tissue 50 to grow into the implant 100. At the same time, however, the dynamic properties of the implant 100 should not be substantially impaired. This can be ensured especially by specifically influencing the ingrowth of the tissue 50.

To this end, FIG. 3.b shows an improvement of the implant 100 shown in FIG. 3.a. The outside surface 10a, in particular at least in a surface region, is designed or treated in such a manner that it allows ingrowth of the tissue 50 into the outside surface 10a of the implant 100, in particular in the surface region mentioned. The core or the inside space of the molded component 11 is to remain substantially free from tissue 50 in order to maintain the dynamic properties of the implant 100 intact.

This can be accomplished, for example, in that the wires 12 that are located on the outside surface 10a of the molded component 11 have a surface different from that of the wires 12 that are located in the core of the molded component 11. To illustrate this, FIG. 3.b schematically shows the implant 100 seen in FIG. 3.a with a different outside surface 10a. One possibility is a wire 12 coated with a coating 14.

For example, titanium or a titanium alloy makes it possible for tissue 50 to grow into a porous structure. In contrast, stainless steel and certain plastic materials prevent or impede ingrowth of the tissue 50. Thus, for example, two possible combinations are titanium/stainless steel and titanium/plastic material. In one embodiment of the present invention, the coating 14 is applied to the finished molded component 11. In this case, the molded component 11 already has its final appearance. More specifically, the outside surface 10a is treated preferably down to the depth desired.

In a first embodiment, the wire 12 of the molded component 11 is made of a material, such as stainless steel, which inhibits ingrowth of the tissue 50. By coating the wire area that forms the outside surface 10a of the implant 100 with a material, for example, titanium or a titanium alloy, it is possible for the tissue 50 to grow into the outside surface 10a of the implant 100. Coating can be deposited by a gaseous state process, for example, by evaporation, by a liquid or pasty state process, for example, by spraying, and/or by electrochemical deposition from solutions, for example, by electroplating. Electroplating is especially useful since the depth and/or the thickness of the coating 14 in the implant can be determined by way of the depth of immersion and/or the length of time the implant 100 or the outside surface 10a of the implant 100 is immersed in a solution.

In a second embodiment, the wire 12 of the molded component 11 is made of a material, for example, titanium or a titanium alloy, which allows ingrowth of the tissue 50. In this case, the wire 12 as such is covered, preferably even prior to knitting, with a layer or a coating 14 of a material, for example, stainless steel or a plastic material, which suppresses ingrowth of the tissue 50. By removing the layer or the coating 14 in the region that forms the outside surface 10a of the implant 100, it is possible for the tissue 50 to grow into the outside surface 10a of the implant 100. The layer or coating can be removed, for example, my means of etching. Etching is especially useful since the depth of the exposed region 14, in this case titanium or the exposed titanium alloy, in the molded component 11 can be determined by way of the depth of immersion and/or the length of time the implant 100 or, more specifically, the outside surface 10a of the implant 100 is immersed in an etching solution or liquid.

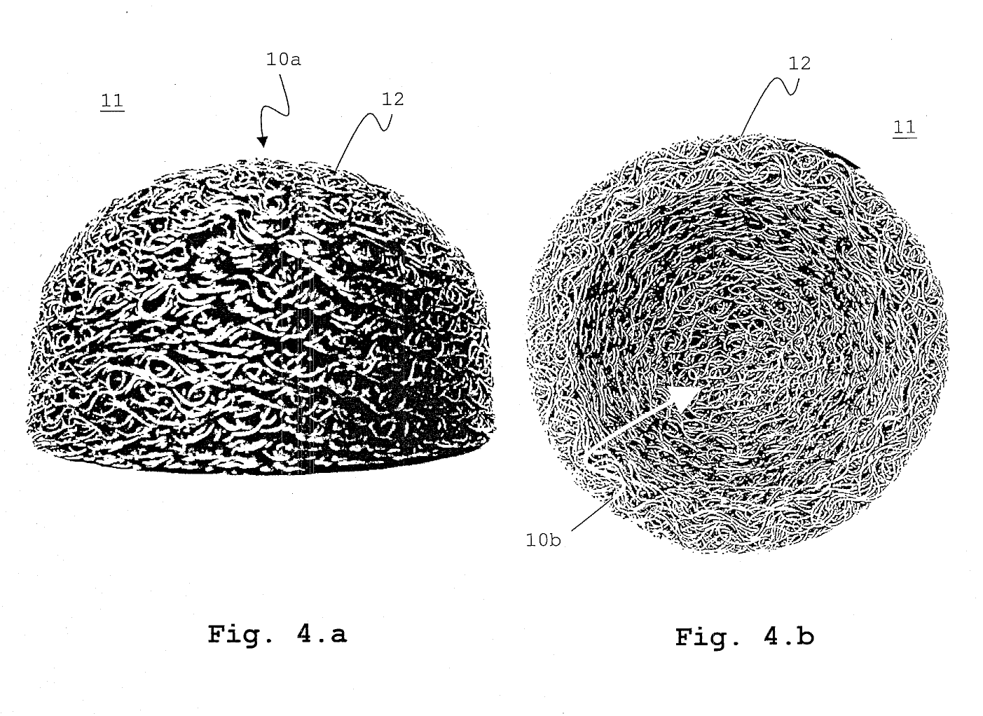

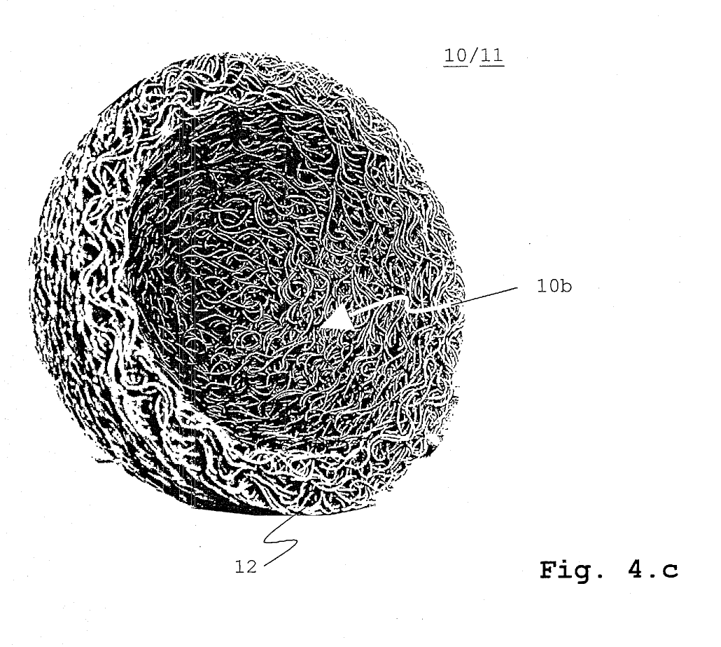

FIGS. 4.a to 4.c show detailed representations of, respectively, a lateral view, a bottom view and a perspective view of a molded component 11. Clearly visible in these figures are the wires 12 of the molded component 11 which, in a preferred embodiment of the invention, initially have the form of a circular knit mesh fabric. In a second step, the mesh fabric is folded and/or rolled up. The folding and/or rolling step is carried out in a manner to accommodate the shape and/or the density of the mesh fabric desired for the finished molded component 11. In the next step, the molded component 11 is shaped by compression molding the mesh fabric which has preferably been folded and rolled up prior thereto.

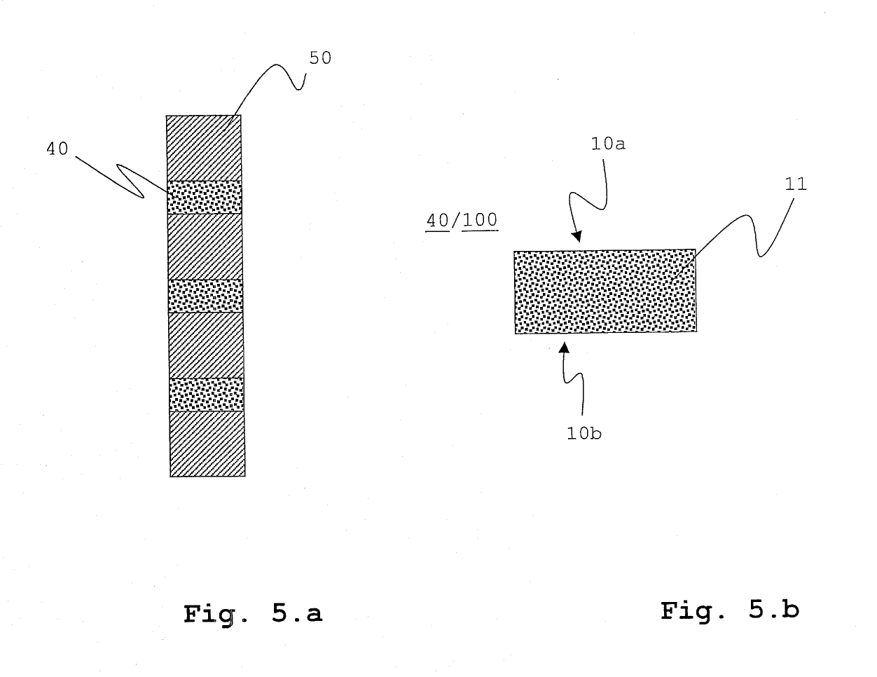

FIGS. 5a and 5b show a section of a vertebral column and an implant 100 for an intervertebral disk 40. The upper surface 10a and the lower surface 10b of the implant 100 rest against the neighboring vertebrae 50. Preferably the molded component 11 alone constitutes the entire implant 100.

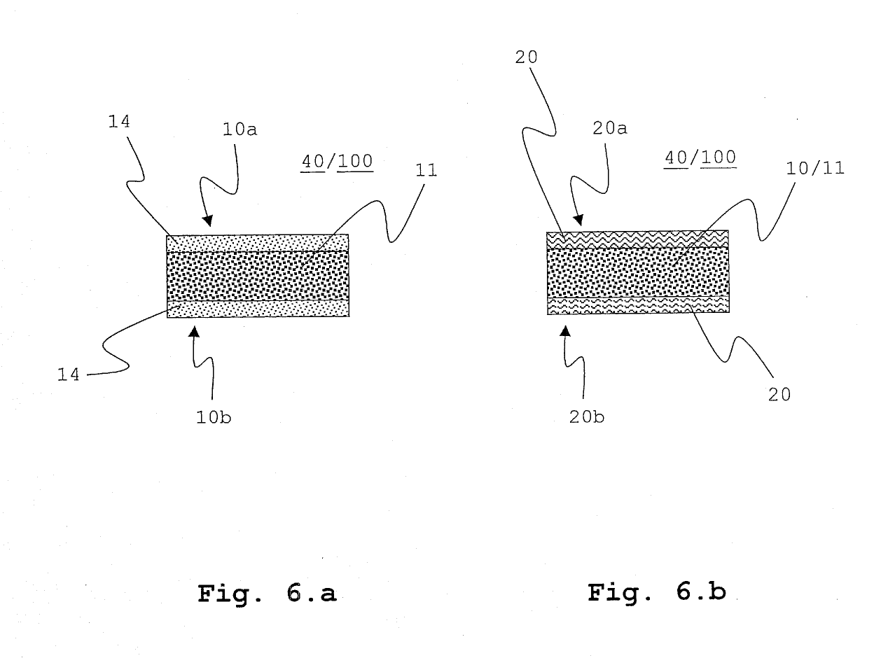

To enable a defined growth of the tissue 50 or the bone 50 into the surface of the implant, both the upper surface 10a and the lower surface 10b of the implant are appropriately configured, preferably down to a desired depth or in a surface region. FIG. 6.a shows a schematic view of a cross section through an implant 100 designed for an intervertebral disk 40 according to the present invention. Reference numeral 14 designates a coating or an exposed area. For details about the structure of the upper surface 10a and the lower surface 10b, reference is made to the description in connection with FIG. 3.b since this description applies to the presently discussed figure as well.

FIG. 6.b shows a cross section through another embodiment of an implant 100 for an intervertebral disk. In this case, a second, especially a nonmetal, section 20 is disposed on the upper surface 10a and the lower surface 10b of the molded component 11, here on the first component 10. The two second sections 20 are the interface or the transitional region to the neighboring tissue 50 or bone 50. The molded component 11 as such is, so to speak, disposed as an elastic core between the two second structures 20. Thus, it is possible to largely prevent the tissue 50 or the bone 50 from growing into the molded component 11 and to maintain the dynamic properties of this component intact. The two second structures 20 can, for example, also have a porous structure, in particular along their upper surface 20a and lower surface 20b in order enable or promote ingrowth of the tissue 50 and thus a stable anchorage. Preferably, the two second structures 20 are made of a plastic material and are formed by spraying them onto the molded component 11.

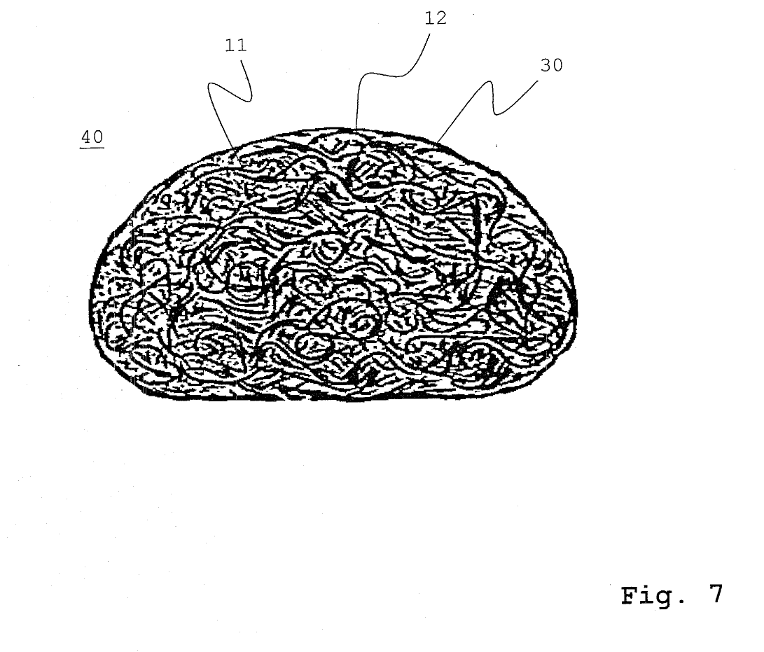

FIG. 7 finally shows a detailed top view of an implant 100 for an intervertebral disk 40 according to the present invention. Again, this figure clearly shows the wires 12 and the porosity of the molded component 11. For greater detail, reference is made to the description in connection with FIGS. 4.a to 4.c.

It will be obvious to the person skilled in the art that the embodiments described are merely offered as examples. The present invention is not limited to these examples and can be varied in many different ways without departing from the scope of the invention. Features of individual embodiments and the features mentioned in the general part of the description can be combined among and with one another.

### LIST OF REFERENCE NUMERALS

10 First section or structure
10a Outside surface or upper surface of the first section or of the molded component
10b Inside surface or lower surface of the first section or of molded component
11 Molded component
12 Wire
13 Shell or hemispherical shell
14 Coating or layer or exposed area
20 Second section
20a Outside surface or upper surface of the second section
20b Inside surface or lower surface of the second section
30 Joint or hip joint
31 Joint socket or acetabulum
31a Outside surface of the acetabulum
31b Inside surface of the acetabulum
31c Inside space of the acetabulum
32 Articular head
33 Stem
34 Intervertebral disk
50 Bone or tissue
100 Implant

1. An implant (100) comprising a composite structure made of
a first section (10) which is formed by a pressure-molded component (11) made of wire (12) and
a second section (20), with the first section (10) being at least in parts embedded in the second section (20).

2. (canceled)

3. The implant as in claim 1, characterized in that the second section (20) is made of a nonmetal material.

4. The implant as in claim 1, characterized in that the second section (20) contains at least one material from the group comprising ceramics and plastic materials.

5. (canceled)

6. The implant as in claim 1, characterized in that the material used to create the second section (20) is sprayed onto the first section (10) and/or in that the material used to create the second section (20) is sintered onto the first section (10).

7. (canceled)

8. The implant as in claim 1, characterized in that the first section (10) is designed as a type of first shell (13) with an inside surface (10b) and an outside surface (10a).

9. The implant as in claim 1, characterized in that the second section (20) is disposed on an inside surface (10b) of the first section (10) or on an outside surface (10a) of the first section (10).

10. The implant as in claim 1, characterized in that the second section (20) is designed as a type of second shell (23) with an inside surface (20b) and an outside surface (20a).

11. The implant as in claim 1, characterized in that the second section (20) is disposed on a surface of the implant (100) which forms a sliding surface.

12. The implant as in claim 1, characterized in that the first section (10) is disposed on a surface of the implant (100) which, after implantation of the implant (100), borders on the surrounding tissue to which the implant is to be anchored.

13. (canceled)

14. An implant (100) comprising a compression-molded component (11) which is made from a wire (12) made of a first material, with parts of the wire (12) of the molded component (11) having a coating (14) made of a second material.

15. The implant as in claim 14, characterized in that the first material or the second material comprises a material which, after implantation of the implant (100), makes it possible to unite with a bordering tissue (5), and/or in that the first material or the second material comprises a bioinert and/or bioactive material.

16. (canceled)

17. The implant as in claim 14, characterized in that the molded component (11) has a coating (14) on at least one of its surfaces (10a,10b) which, after implantation of the implant (100), rests against a surrounding tissue (50) so that the tissue (50) is able to grow into a surface region of the molded component (11).

18. The implant as in claim 14, characterized in that the molded component (11) does not have coating (14) on at least one of its surfaces (10a,10b) which, after implantation of the implant (100), rests against a surrounding tissue (50) so that the tissue (50) is able to grow into a surface region of the molded component (11).

19. A method for the production of an implant (100) comprising:
making available a mesh fabric made of wire (12),
folding and/or rolling up the mesh fabric,
compression-molding the mesh fabric into a molded component (11) to create a first section (10),
making available a second section (12), and
connecting the first section (10) and the second section (20) with each other, with the first section (10) being at least partially embedded in the second section (20).

20. The method as in claim 19, characterized in that the second section (20) is formed on top of the first section (10).

21. The method as in claim 19, characterized in that at least certain parts of the second section (20) have a viscosity such that it is possible to embed the first section (10) in the second section.

22. The method as in claim 19, characterized in that the second section (20) is sprayed onto the first section (10) and/or in that the second section (20) is sintered onto the first section (10).

23. (canceled)

24. A method for use in the production of an implant (100) comprising:
making available a mesh fabric made of wire (12) of a first material,
folding and/or rolling up the mesh fabric,
compression-molding the mesh fabric into a molded component (11),
depositing at least a partial coating of a second material on the wire (12), with the first material or the second material being a material which, after implantation of the implant (100), allows the tissue (50) to grow into parts of a surface region (10a,10b) of the molded component (11) along a region bordering a surrounding tissue (50).

25. The method as in claim 24, characterized in that, after compression molding, at least one surface (10a,10b) of the molded component (11) is coated so that, after implantation of the implant (100), the coating (14) is able to unite with an adjoining tissue (50).

26. The method as in claim 24, characterized in that, after compression molding, the coating (14) is removed at least on one surface of the molded component (11) so that, after implantation of the implant (100), the exposed surface of the wire (12) is able to unite with an adjoining tissue (5), preferably by allowing the tissue (50) to grow into the molded component (11).
